(19) United States
(12) Patent Application Publication
(10) Pub. No.: US 2014/0309532 A1
(43) Pub. Date: Oct. 16, 2014

(54) Method for non-invasively detecting the narrowing of a lumen

(71) Applicants: Allison Bedell, Winnetka, IL (US); Mohammad-Kasim Hussain Fassia, Grand Rapids, MI (US)

(72) Inventors: Allison Bedell, Winnetka, IL (US); Mohammad-Kasim Hussain Fassia, Grand Rapids, MI (US)

(21) Appl. No.: 14/207,523

(22) Filed: Mar. 12, 2014

Related U.S. Application Data
(60) Provisional application No. 61/777,583, filed on Mar. 12, 2013.

(51) Int. Cl. A61B 8/08; A61B 8/00

(52) U.S. Cl. CPC A61B 8/0858; A61B 8/4494; A61B 8/5223; A61B 8/4477; A61B 8/4209
USPC 600/449; 600/587

(57) ABSTRACT

Disclosed is a method for non-invasively detecting narrowing in a lumen in the body. This is done using sonic signals, e.g. ultrasound, that are directed through the wall and into the lumen of the tube, such that they travel axially and backscatter is returned and read. This method can be used in a number of applications including both naturally-occurring vessels and artificial catheters.

### CROSS-REFERENCE TO RELATED APPLICATIONS

This application claims the benefit of U.S. Provisional Application No. 61/777,583, filed Mar. 12, 2013.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

This invention relates to medical diagnostics, specifically to detecting the narrowing of naturally-occurring or medically-implanted anatomical lumens.

(2) Description of the Related Art

There are many vessels in the body, both naturally-occurring (blood vessels, lymph vessels, bronchial tubes, etc) and implanted (shunts, stents, and other implanted catheters). Many of these vessels are susceptible to failure by clog, kink, disconnect, or other narrowing of the lumen. When the lumen narrows, the internal matter has more difficulty passing through, which can pose significant risk to the patient.

For the remainder of this document, the term tube (20) will be used to refer to anatomical or medically-implanted vessels which provide a conduit for the passage of fluid or gas. The term lumen (21) will be used to refer to the enclosed, hollow region within the tube where the fluid or gas passes through. The term patency will be used to describe a completely open, unobstructed lumen. The term narrowing (22) will be used to describe a decrease in the unobstructed cross-sectional area of the lumen. Narrowing can be caused by partial or full obstructions (e.g. clogs), kinks, disconnections, floating matter that hinders flow through some or all of the lumen, and other disruptions to the patency of the tube. The term tube wall (23) will be used to refer to the layer of material (e.g. tissue, silicone, etc) defining the lumen. The term internal matter (24) will be used to refer to the fluid or gas within or traveling through the lumen. The proximal end of the tube (25) will be used to refer to the beginning of a segment of tube where the internal matter enters the lumen. The distal end of the tube (26) will be used to refer to the end of a segment of tube where the internal matter exits the lumen of the tube. The internal matter travels from the proximal end of the tube to the distal end. Skin (27) will be used to refer to the outermost layer of anatomical tissue, which can be contacted in a noninvasive manner. Tissue (28) will be used to refer to all biological tissue or other anatomical structures between the skin and the tube wall. FIGS. 1, 2, and 3 illustrate these terms.

The following describes the prior art in the space of detecting the narrowing of a lumen.

Endoscopes (like US2012/0179050) are available to tunnel into the human body through existing orifices or surgical incisions. These devices provide clear images visualizing the inside of the lumen. However, these tools are very invasive (posing high risk to the patient) and require the labor-intensive process of manually checking the entire interior of the lumen. Even with these devices, some problematic obstructions can be overlooked.

Ultrasonic catheters (like U.S. Pat. No. 8,535,232) can also be employed by the same method. These catheters are tunneled through the body to visualize or otherwise detect blockage. This, like endoscopes, is an invasive and tedious procedure.

Surgical interrogations will also be performed where a surgeon opens up the lumen and visually examines it. These procedures are extremely invasive and are generally used when alternatives are exhausted. For medical conditions where there are few nonsurgical diagnostic tests, patients will often undergo a series of tests to eliminate other possible causes of their symptoms before they are scheduled for surgery. During this time the patient stays in the hospital, putting them at risk for a hospital-acquired infection.

Ultrasound is also used non-invasively for both its imaging and measurement capabilities. In U.S. Pat. No. 4,261,367, ultrasonic waves are used to non-invasively measure the diameter of a blood vessel. Alternatively, a device as in U.S. Pat. No. 8,409,097 employs ultrasound to visualize the lumen of a tube. While these are much less risky and invasive procedures and subject the patient to an insignificant dose of radiation, these tests require that a sonographer manually search every inch of the tube. They must manipulate the probe to align it so that it can acquire data from every section of the tube. This is labor-intensive and takes time.

X-rays and MRI cannot penetrate the tube wall and therefore will only visualize the outside of the tube wall and its surrounding area. This can be useful to see kinks and disconnections but cannot see internal clogs.

Sometimes MRI and CT scans are used to visualize the region of the body surrounding the proximal end of the tube to see if there is a buildup of the internal matter at that location, indicating a narrowing downstream. However, oftentimes this buildup can only be seen after enough pressure has built up to do damage to the body.

Sensing the flow or pressure of the internal matter may also be employed. This can be done using an implantable sensor like in US2009/0143673 or it can be non-invasively measured like in U.S. Pat. No. 8,328,727. A decrease in flow or increase in pressure can suggest a narrowing in the line. In many applications, like shunting, a change in either of these parameters can occur under normal conditions without any compromise to the patency of the lumen. Even when a change in flow or pressure is proven to directly correlate with a narrowed lumen, no information is given about the location or cause of the restricted flow or increased pressure. This makes resolving the issue more difficult.

The preferred embodiment of the disclosed invention is for use with shunts. Hydrocephalus patients often require shunts to facilitate the drainage of cerebral spinal fluid (CSF) from the ventricles of the brain to the peritoneal cavity to relieve pressure building up in the brain due to excess CSF. While implanting a shunt provides an effective treatment for hydrocephalus, these shunts are prone to blockage and the symptoms that present when this happens are not unique enough to make a diagnosis based on them alone. Methods like those mentioned above are currently employed for this application. However, invasive surgery is the only truly conclusive test for shunt catheter lumen patency. Given the high risk of infection associated with invasive surgery and a prolonged hospital stay, the need for a non-invasive test for shunt blockage is apparent.

Generally, the present invention provides a way to non-invasively detect blockage or the otherwise narrowing of a lumen, e.g. a shunt catheter. This method delivers no significant radiation to the patient and can be used to search the entire lumen at once.

### BRIEF SUMMARY OF THE INVENTION

Disclosed is a sonic-based method for noninvasively detecting the narrowing of a lumen. This method does not expose the patient to significant radiation or invasive operations. The signal generator and emitter will together produce sonic waves, e.g. ultrasound, and they will be directed through the skin and tissue, through the tube wall, and into the desired lumen. The device will include a receiver element that will read and interpret the backscatter of the transmitted signal in order to evaluate the lumen to differentiate between sites of patency and sites of narrowing.

### BRIEF DESCRIPTION OF THE SEVERAL VIEWS OF THE DRAWINGS

FIG. 1 illustrates the anatomy where the embodiment of this device is intended to be used

FIG. 2 illustrates a cross section of a patent tube

FIG. 3 illustrates a cross section of a tube with a narrowed lumen

FIG. 4 illustrates how a sonic signal will refract when directed into the anatomy illustrated in FIG. 1.

FIG. 5 shows a block diagram describing the basic components required for a sonic system

FIG. 6 illustrates the event when there is a narrowing in the lumen and what the acquired signal will look like

FIG. 7 illustrates the event that there is a patent lumen, that is such that there will be backscatter from the end of the lumen, and what the acquired signal will look like

FIG. 8 illustrates the event that there is a patent lumen, which is such that there is no backscatter from the end of the lumen, and what the acquired signal will look like

FIG. 9 illustrates the preferred embodiment, with a positioning element that allows for a variable angle

FIG. 10 illustrates an alternative embodiment, with a positioning element that remains at a fixed angle

FIG. 11 illustrates an alternative embodiment, with two emitters and two receiver elements pointed in opposite directions.

### DETAILED DESCRIPTION OF THE INVENTION

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

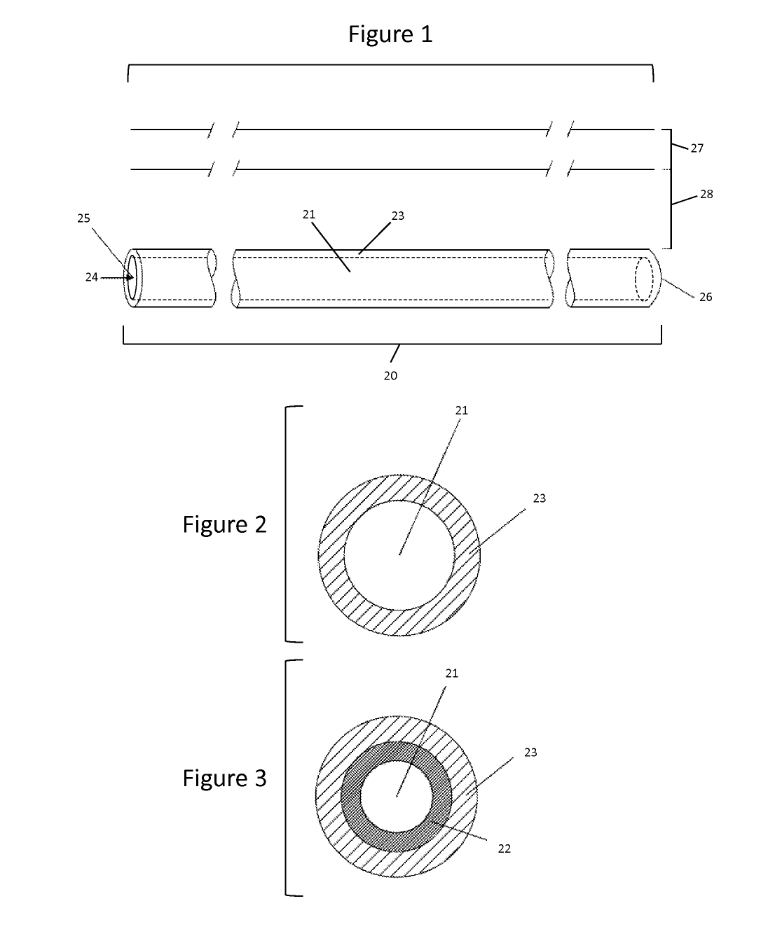

FIGS. 1, 2 and 3 illustrate an example of a lumen. FIG. 2 shows a patent lumen and FIG. 3 shows a narrowed lumen. The illustrated parts are described in the background section of this document.

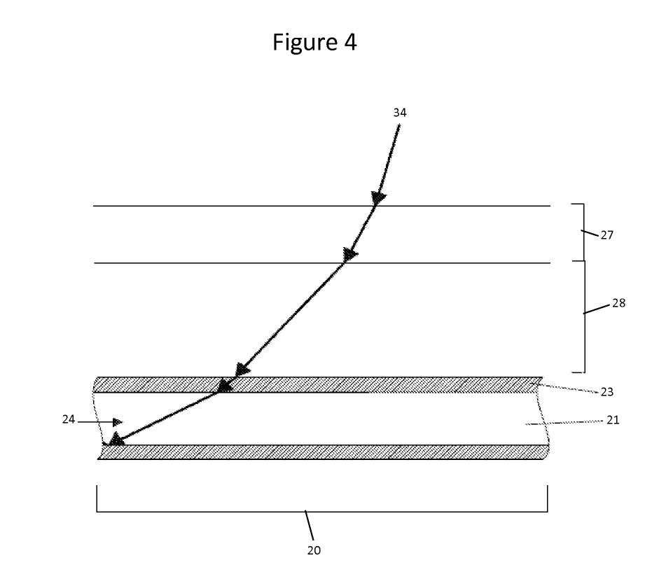

FIG. 4 illustrates how a sonic signal (34) may behave when introduced to the environment in FIG. 1. This signal shows one example of how a signal may refract through various material interfaces but this will change based on the application and materials involved.

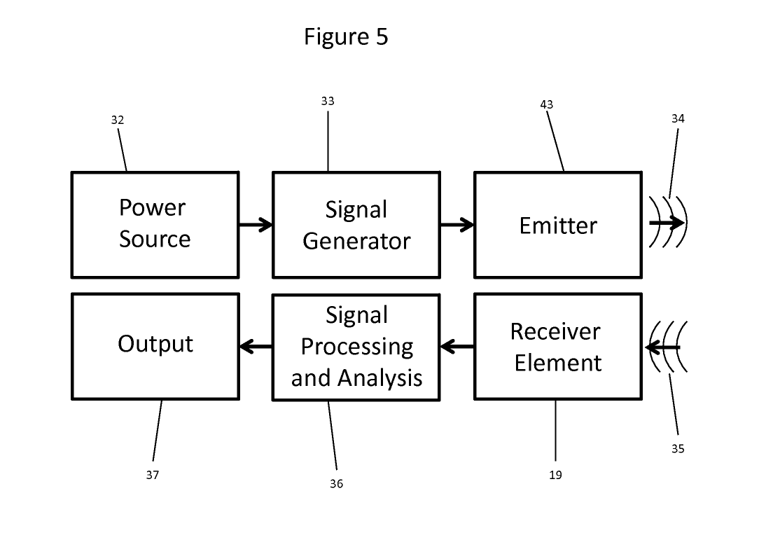

FIG. 5 shows a block diagram of the necessary components to produce and interpret sonic waves. First, a power source (32) provides the necessary energy for a signal generator (33) to create a signal that the emitter (43) converts into an emitted sonic wave (34). The returned signal (35) is returned to the receiver element (19) which acquires the signal. This signal is filtered and interpreted by the signal processing and analyzing functions (36). Finally this analyzed signal or the information extracted from it becomes the output (37) which is given to the user.

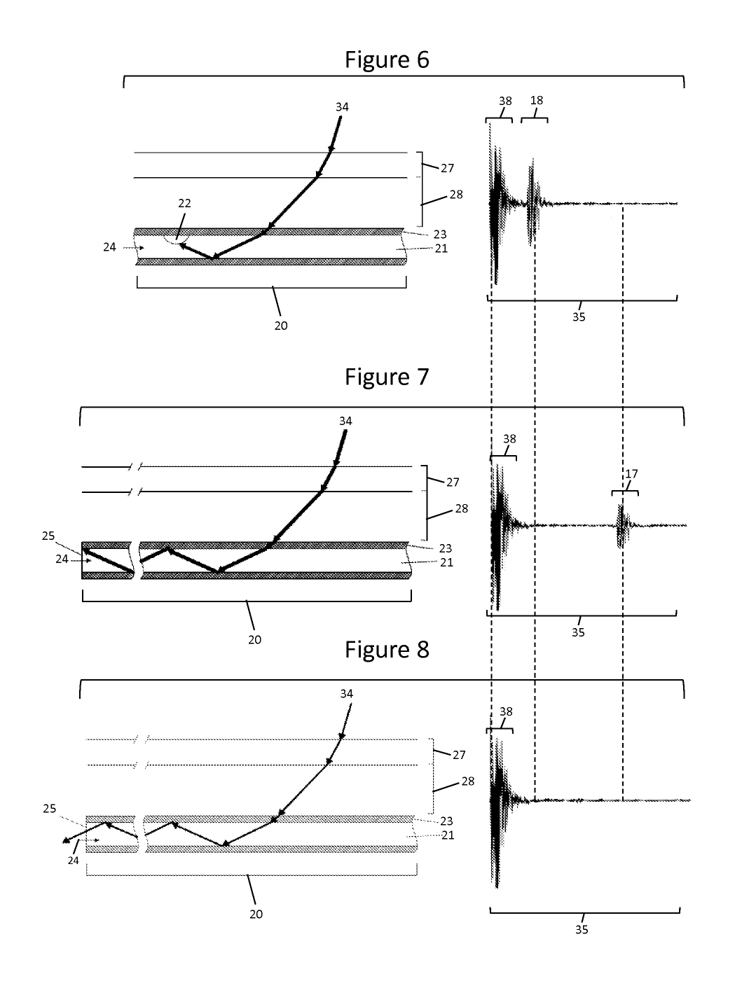

FIGS. 6 and 7 show the two most common outcomes of applying the sonic signal (34) to the lumen. In FIG. 6, there is a blockage that narrows the lumen (22) and the acquired signal (35) reveals the presence of this obstruction by the returned backscatter (18). The other part of the signal (38) is an artifact of the sent signal. The time elapsed between these two waveforms can be used to calculate the distance to the obstruction. In FIG. 7, the lumen is patent and the backscatter (17) is received from the proximal or distal end of the tube. This is also what a baseline signal may look like.

In a third possible outcome, FIG. 8, there may be no backscatter received and the acquired signal will only show an artifact of the sent signal (38). This will occur in the event that the lumen is patent and no backscatter is received from the proximal or distal end of the tube.

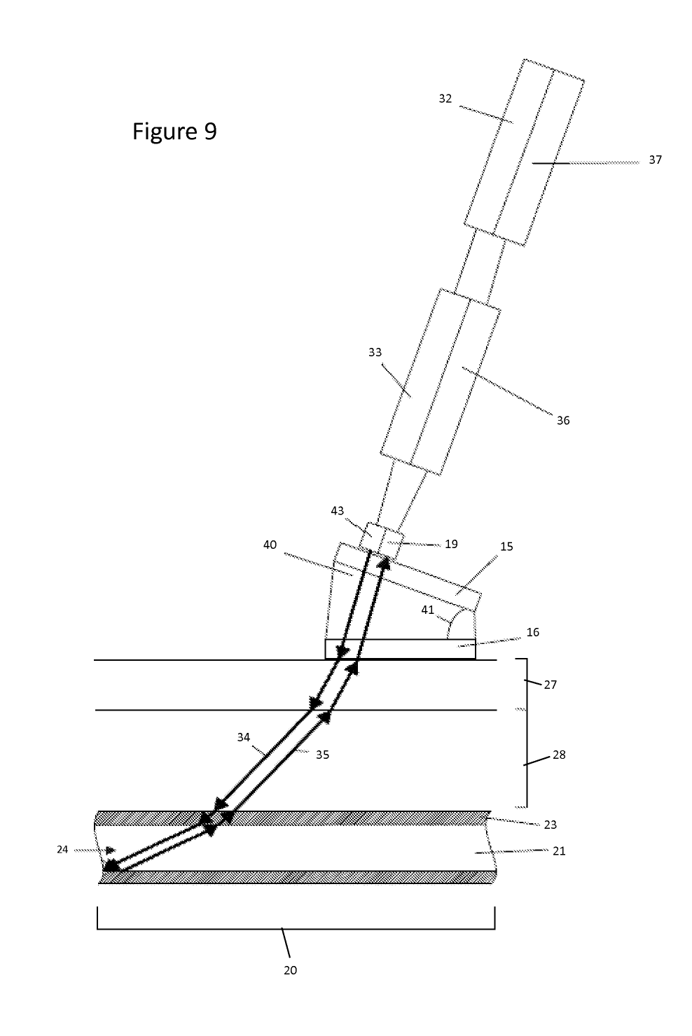

FIGS. 9, 10, and 11 illustrate embodiments of the device. FIG. 9 is the preferred embodiment but any combination of the features described can create a feasible embodiment.

FIG. 9 shows a preferred embodiment of the device. This embodiment consists of at least one emitter (43) and receiver element (19), positioned against a solid object (40) consisting both of a first surface (16) that sits against the skin and a second surface (15) that can be orientated in many directions relative to the first surface to create angles, θ (41), between 0 and 90 degrees with the first surface. The object is made of a material that transmits sonic signals well, like rubber, plexiglass, or rexilite. The object can also be positioned such that it can maintain an established angle. It may also return to this angle when prompted. The object (40) is the positioning element.

In the preferred embodiment of this device the decision about whether backscatter is significant is made by an internal algorithm (36) or an algorithm within a software that compares a baseline signal to the current acquired signal. The baseline signal may be of a lumen that is known to be patent, possibly from the same patient (e.g. at time of implantation) or of another patient with the same kind of lumen.

Also, in the preferred embodiment of this device the output (37) is a list or visualization describing locations where significant backscatter (18) was returned to the receiver element (19), and the severity of these backscatter signals. It is reasonable to suspect narrowing of the lumen at those locations.

Very basically, the location of a narrowed segment of lumen can be calculated by taking the acquired waveform (35) and measuring the time elapsed between the artifact of the emitted signal (38) and the recorded backscatter (18) and multiplying it by the known velocity of sonic signals when propagating through a tube of identical internal matter. Other factors are also used like the depth of the lumen in the body and the diameter of the lumen.

The degree to which the lumen is narrowed (to which the flow of the internal matter is hindered) will be calculated by comparing the relative strength of backscatter signals. Backscatter of great strength will be acquired from more constricted locations.

In the preferred embodiment of this device, the backscatter at various times and from various transducers will be averaged over time to eliminate noise.

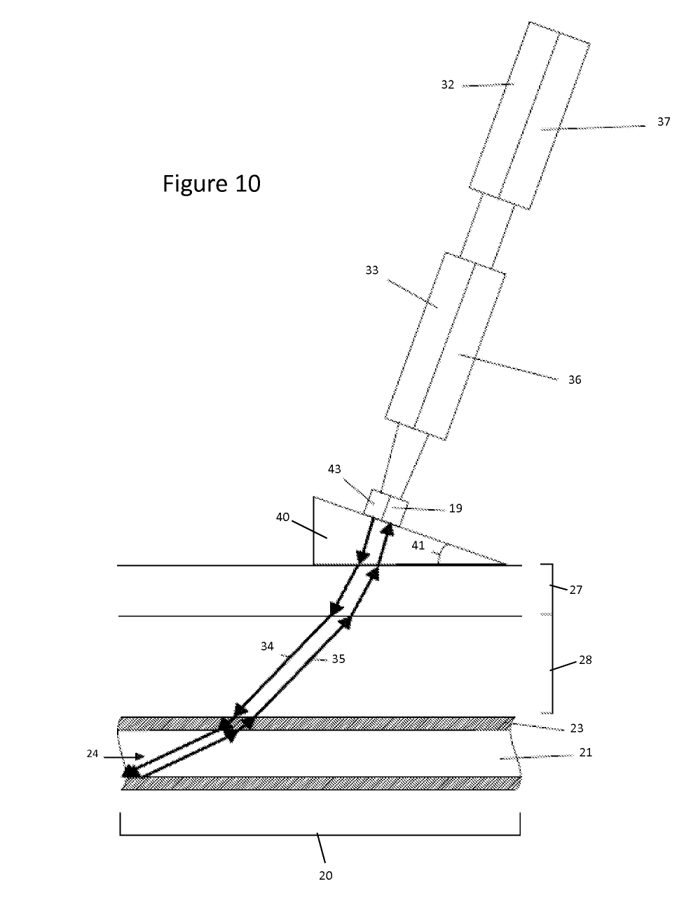

FIG. 10 shows another embodiment of this device. This embodiment consists of a solid object (40) consisting of two fixed planar surfaces, one where the transducer is placed and one that is placed against the skin. Between these two surfaces is a fixed angle, θ (41), which will be the set angle for a given application. This angle will be predetermined based on factors like the location of the lumen relative to the skin and the composition of the surrounding anatomy.

In said embodiment the decision about whether the backscatter indicates the presence of narrowing lumen is made by an internal algorithm (36) or software that reports backscatter with a change in signal strength above a certain user-set or user-selected preset threshold. The threshold for a particular application may be determined by a study correlating backscatter to a known obstruction at a known location.

In said embodiment the output (37) is an indication about whether there exists a significant narrowing or not, without any information given about the location or severity of these instances of narrowing.

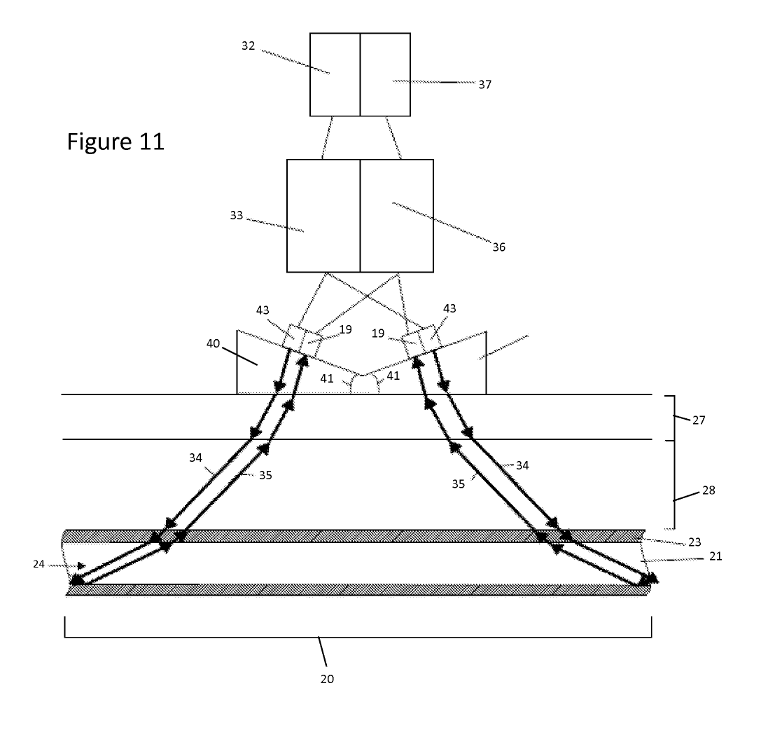

FIG. 11 shows another embodiment of this device where the emitters (43) and receiver elements (19) are split into two groups, each containing half of the emitters and half of the receiver elements. Each group faces in an opposite direction. In this embodiment, information about the lumen in either direction is obtained and separately analyzed using any of the methods described above. Again, this method can employ an object (40), which serves as the positioning element, with a fixed angle (41) or one that allows for a variable angle.

In said embodiment, the acquired signal (35) is outputted to the user, and the user has control over the signal processing and analysis process. Said tools may consist of a number of filters and thresholds that may be applied to the signal in an analog or digital form to make the signal more readable. The user shall make a decision about whether there is indication of a narrowing lumen.

In another embodiment both the list or visualization of locations and the acquired signal are given to the user to make an even more informed decision.

1. A method for detecting the narrowing of a lumen in the body, based on:
a. A signal generator
b. An emitter(s)
c. A receiver element(s)

2. The method of claim 1 wherein the emitter produces waves of an ultrasonic frequency

3. The method of claim 1 wherein the emitter consists of one transducing element

4. The method of claim 1 wherein the emitter consists of a plurality of transducing elements arranged in an array

5. The method of claim 1 wherein the emitter consists of a plurality of transducing elements pointed in different directions relative to the skin

6. The method of claim 1 wherein said emitter and said receiver element are the same element.

7. The method of claim 1 wherein the signal acquired by the receiver element is processed by analog or digital means of signal processing

8. The method of claim 1 wherein the signal acquired by the receiver element is analyzed by comparing it to a baseline signal

9. The method of claim 8 wherein the baseline signal is a signal acquired from a lumen that is known to be patent

10. The method of claim 9 wherein said baseline signal is a signal acquired from the same patient as the current acquired signal

11. The method of claim 1 wherein the signal acquired by the receiver element is compared to a relative threshold to detect narrowing of the lumen

12. The method of claim 11 wherein said threshold is different for every use

13. The method of claim 1 wherein the signal acquired by the receiver element is processed to determine the location of the narrowing using the time delay between the signal sent from the emitter and the acquired signal received by the receiver element

14. The method of claim 1 wherein the positioning element is composed of a material that does not dissipate the signal

15. The method of claim 1 further based on a positioning element

16. The method of claim 15 wherein the positioning element holds the emitter and receiver element at a fixed angle relative to the skin

17. The method of claim 16 wherein the fixed angle of the positioning element is variable and can be adjusted by the user

18. A method for detecting the narrowing of a lumen inside the body comprising the steps of:
a. Directing one or more sonic waves into a lumen such that they travel axially down the lumen
b. Acquiring the backscatter of these waves
c. Interpreting the backscatter to determine whether at any point along the lumen there exists a narrowing in the lumen

19. The method of claim 18 wherein further comprising the step of analyzing the backscatter to determine the location of the narrowing

20. The method of claim 18 wherein further comprising the step of analyzing the backscatter to determine the severity of the narrowing.
